(19) United States
(12) Patent Application Publication
(10) Pub. No.: US 2008/0032946 A1
(43) Pub. Date: Feb. 7, 2008

(54) METHOD FOR PRODUCING A COMPOSITE MADE OF OLIGONUCLEOTIDES OR POLYNUCLEOTIDES AND HYDROPHOBIC BIODEGRADABLE POLYMERS AS WELL AS COMPOSITE OBTAINED ACCORDING TO THE METHOD

(75) Inventors: Alexander Borck, Aurachtal (DE); Tobias Diener, Erlangen (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/831,627

(22) Filed: Jul. 31, 2007

(30) Foreign Application Priority Data
Aug. 7, 2006 (DE) 10 2006 038 240.4

(51) Int. Cl. C07H 21/00; A61K 31/7088

(52) U.S. Cl. 514/44; 536/23.1; 536/25.3

(57) ABSTRACT

A method for producing a composite made of oligonucleotides or polynucleotides and hydrophobic biodegradable polymers. The method comprises the following steps: (i) producing an aqueous solution of a polyethylene glycol having a mean molecular weight in the range from 600 to 8,000 g/mole; (ii) adding an aqueous, possibly buffered solution of oligonucleotides or polynucleotides to the aqueous solution from step (i); iii) freezing the aqueous solution from step (ii) and subsequently freeze-drying the frozen solution to obtain a freeze-dried substrate; (iv) providing a solution of the hydrophobic biodegradable polymer in an organic solvent; (v) adding the substrate obtained according to step (iii) to the solution of the polymer; and (vi) removing the organic solvent to obtain the composite. Also disclosed is a composite produced by the method.

### PRIORITY CLAIM

This patent application claims priority to copending German Patent Application No. 10 2006 038 240.4, filed Aug. 7, 2006, the disclosure of which is incorporated herein by reference in its entirety.

### FIELD

The present disclosure relates to a method for producing a composite made of oligonucleotides or polynucleotides and hydrophobic biodegradable polymers, a composite obtained according to the method, and a use of the composite.

### BACKGROUND

Oligonucleotides and polynucleotides are increasingly used as therapeutic active ingredients. It is typically not possible to absorb the active ingredients parenterally; rather, the active ingredients must be administered interstitially. One problem in developing a suitable medication is the low solubility of oligonucleotides and polynucleotides in hydrophobic biodegradable polymers. However, for the purposes of interstitial administration, the biodegradable polymers represent especially suitable carrier systems. Because of the hydrophobic matrix properties, preparing homogeneous composites, in which the nucleotides are finely distributed, is made more difficult; and, in addition, composites of this type are not sufficiently storage stable, because recrystallization of the nucleotides initially provided in amorphous modifications occurs. The recrystallization changes the release behavior of the nucleotides from the biodegradable polymer matrix so that, in the extreme case, the intended treatment may no longer be followed.

### SUMMARY

The present disclosure provides several exemplary embodiments of the present invention, some of which are discussed below.

One aspect of the present disclosure provides a method for producing a composite made of oligonucleotides or polynucleotides and hydrophobic biodegradable polymers, comprising the steps of (i) producing an aqueous solution of a polyethylene glycol having a mean molecular weight in the range from 600 to 8,000 g/mole; (ii) adding an aqueous, possibly buffered, solution of oligonucleotides or polynucleotides to the aqueous solution from step (i); (iii) freezing the aqueous solution from step (ii) and subsequently freeze-drying the frozen solution to obtain a freeze-dried substrate; (iv) providing a solution of the hydrophobic biodegradable polymer in an organic solvent; (v) adding the substrate obtained according to step (iii) to the solution of the polymer; and (vi) removing the organic solvent to obtain the composite.

Another aspect of the present disclosure provides a composite obtained or obtainable according to a method comprising the steps of (i) producing an aqueous solution of a polyethylene glycol having a mean molecular weight in the range from 600 to 8,000 g/mole; (ii) adding an aqueous, possibly buffered, solution of oligonucleotides or polynucleotides to the aqueous solution from step (i); (iii) freezing the aqueous solution from step (ii) and subsequently freeze-drying the frozen solution to obtain a freeze-dried substrate; (iv) providing a solution of the hydrophobic biodegradable polymer in an organic solvent; (v) adding the substrate obtained according to step (iii) to the solution of the polymer; and (vi) removing the organic solvent to obtain the composite.

A further aspect of the present disclosure provides a composite useful as a coating material for a medical implant or as a filler of a cavity of a medical implant, the composite produced according to a method comprising the steps of (i) producing an aqueous solution of a polyethylene glycol having a mean molecular weight in the range from 600 to 8,000 g/mole; (ii) adding an aqueous, possibly buffered, solution of oligonucleotides or polynucleotides to the aqueous solution from step (i); (iii) freezing the aqueous solution from step (ii) and subsequently freeze-drying the frozen solution to obtain a freeze-dried substrate; (iv) providing a solution of the hydrophobic biodegradable polymer in an organic solvent; (v) adding the substrate obtained according to step (iii) to the solution of the polymer; and (vi) removing the organic solvent to obtain the composite.

### DETAILED DESCRIPTION

It has been shown that composites produced according to the method of the present disclosure are significantly more storage-stable and have a very homogeneous distribution of the nucleotides. A conjugate made of polyethylene glycol and the nucleotides apparently forms in steps (i) through (iii). This conjugate also appears to have stability in the further method steps and is also equipped with the desired effects of improved solubility and storage stability. It has been shown that polyethylene glycol may assume the role of a solution mediator, so that it is assumed that polyethylene glycol molecules envelope the nucleotides in the conjugate. The conjugate apparently separates again only slightly, or not at all, upon solution in the organic solvent; rather, a thermodynamically favorable state appears to be reached. The significantly better solubility of the conjugate than the pure nucleotides allows the production of highly homogeneous composites. These composites additionally have significantly greater storage stability, apparently because of the stabilizing effects of the polyethylene matrix.

In step (i) of the method, an aqueous solution is produced from a polyethylene glycol having a mean molecular weight in the range of 600 to 8,000 g/mol.

Polyethylene glycol (PEG) is a name for polyalkylene glycols belonging to the class of polyethers. Polyethylene glycols are non-ionogenic compound of the general formula:

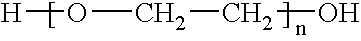

Polyethylene glycols are industrially produced by anionic ring opening polymerization of ethylene oxide (oxirane), usually in the presence of small quantities of water (or also sodium methylate or alkali hydroxide). They have molar masses in the range from approximately 200 to 5,000,000 g/mole depending on the reaction control, corresponding to degrees of polymerization Pn of approximately 5 to greater than 100,000. Polyethylene glycols are liquid or wax-like to solid products, which dissolve well in water up to approximately 100° C. and in many organic solvents. Polyethylene glycols are largely physiologically inert and are classified as toxicologically harmless. Their biological decomposability is strongly dependent on their molar mass; products having low molar masses, e.g., 4,000 g/mole, are decomposed up to 80%. In pharmaceutical technology, polyethylene glycols are used as solvent mediators, softeners, ointment bases, emulsifiers, lubricants, and mold lubricants in tablet production and as components of dragee suspensions.

Polyethylene glycols having a mean molecular weight in the range from 600 to 8,000 g/mole are used. Polyethylene glycols in the cited molecular weight range display a sufficiently high water solubility. Polyethylene glycols having a molecular weight of less than 600 g/mole have a pronounced hydrophilic character, so the polyethylene glycols do not appear suitable as solution mediators. The formation of a conjugate is possibly also thermodynamically less favorable in the event of relatively low chain links of the polyethylene glycol. Polyethylene glycols having a molecular weight of greater than 8,000 g/mole, in contrast, appear to have a character too strongly hydrophobic, so that the formation of a conjugate with the nucleotides is made more difficult. The chain length is possibly also too great for the formation of such a conjugate.

The aqueous solution from step (i) preferably contains polyethylene glycol in concentrations from 0.1 to 5 g/l polyethylene glycol. The concentration is especially preferably 0.5 to 1 g/l. It has been shown that working outside the cited range boundaries apparently makes the formation of the composite more difficult.

In step (ii) of the method an aqueous, possibly buffered, solution of oligonucleotides or polynucleotides is added to the aqueous solution from step (i). Oligonucleotides or polynucleotides typically have good water solubility and are stored in buffered solutions for stabilization. The solution preferably contains the nucleotides in a concentration in the range from 0.1 to 7 μg/μl.

A quantity ratio of oligonucleotides or polynucleotides to the polyethylene glycol is preferably established so that polyethylene glycol is provided in 1.2 to 100 fold gravimetric excess.

Oligonucleotides are polymers made of approximately 2 to 10 mononucleotides, which are linked by phosphoric acid diester bridges. Oligonucleotides arise upon the cleavage of polynucleotides and nucleic acids or by synthesis, e.g., according to the solid phase technique. Oligonucleotides are used, for example, as antisense inhibitors of gene expression. Polynucleotide is a collective name for polymers which are constructed from more than 10 nucleotide units linked by 3′,5′ phosphodiester bridges. The polynucleotides include the naturally occurring nucleic acids above all, but also synthetic products. The oligonucleotides or polynucleotides are preferably provided as DNA fragments, in particular, dODN. The oligonucleotide or polynucleotide may be provided as a single or double strand.

In step (iii) of the method, the aqueous solution from step (ii) is first frozen. Subsequently, the frozen solution is freeze-dried, a freeze-dried substrate precipitating. This substrate may have a consistency like cotton wool.

In step (iv) of the method, a solution of the hydrophobic biodegradable polymer in an organic solvent is provided. Preferably, the organic biodegradable polymer is a polylactide, in particular a poly-L-lactide.

Polylactides are polyesters based on lactic acid, from whose lactide they may be produced by ring opening polymerization. Polylactide, as a polyhydroxycarboxylic acid, is biologically decomposable and is used, inter alia, as a resorbable surgical suture material and as a capsule material for pharmaceuticals.

Suitable organic solvents comprise acetone, ethanol, ethyl acetate, acetyl acetone, hexafluoroisopropanol, THF, dichloromethane, combinations thereof and the like.

The organic solvent is preferably chloroform.

The solvent preferably contains the hydrophobic biodegradable polymer in a concentration in the range from 0.5 to 7.5 g/liter.

In step (v) of the method, the substrate obtained according to step (iii) is added to the solution of the polymer. The addition is performed while stirring the solution to achieve the most homogeneous distribution possible of the substrate to be dissolved. The hydrophobic biodegradable polymer is preferably provided in 1 to 80 fold gravimetric excess to the oligonucleotide or polynucleotides.

In step (vi) of the method, the organic solvent is removed to obtain the desire composite. Typically, the organic solvent is withdrawn under reduced pressure at room temperature, to avoid thermally induced metabolization of the nucleotides.

A second exemplary aspect is directed to composites which are obtained or obtainable according to the method described above.

Finally, a third exemplary aspect is directed to the use of such a composite as a coating material for a medical implant or as a filler of a cavity of the medical implant.

For purposes of the present disclosure, implants are devices introduced into the body via a surgical method and comprise fasteners for bones, such as screws, plates, or nails; intestinal clamps; vascular clips; prostheses in the area of the hard and soft tissue; anchoring elements for electrodes, in particular, of pacemakers or defibrillators, combinations thereof and the like.

The implant is preferably a stent. Stents of typical construction have a filigree support structure made of metallic struts, which are first provided in a non-expanded state for introduction into the body and which are widened into an expanded state at the location of application. Because of the small size of the areas suitable for coating and/or volumes of cavities, relatively highly concentrated composites must be used to correspond to the therapeutic settings at the location of implantation. Furthermore, stents are typically stored, i.e., the coating/filling must also be storage-stable over a specific period of time. Highly concentrated matrices containing nucleotides may be implemented with the aid of the composite, which additionally have the required storage stability.

Solutions of the following compositions were produced:

### Solution 1: A solution of 1 g polyethylene glycol having a mean molecular weight in the range from 4,000 to 6,000 g/mole in 500 ml distilled water.

### Solution 2: A solution of a dODN having a molecular weight of 10,000 g/mole in a buffered aqueous solution, included as a buffer.

### Solution 3: A solution of poly-L-lactide 1.5 g having a mean molecular weight in the range from 94,000 to 700,000 g/mole in 200 ml chloroform.

0.5 ml of solution 2 was added at room temperature and while stirring to 500 ml of solution 1. The solution obtained was then cooled to −25° C. and the frozen solution obtained was mechanically pulverized and freeze-dried under the following conditions:

The freeze-drying occurred under reduced pressure (3×10⁻⁶ bar) over 24 hours to ensure that the water was completely removed.

The substrate obtained after the freeze-drying was added to 200 ml of solution 3 while stirring and at room temperature. The organic solvent was subsequently withdrawn under reduced pressure at room temperature.

All patents, patent applications and publications are incorporated by reference herein in their entirety.

Claimed is:

1. A method for producing a composite made of oligonucleotides or polynucleotides and hydrophobic biodegradable polymers, comprising the steps of:
(i) producing an aqueous solution of a polyethylene glycol having a mean molecular weight in the range from 600 to 8,000 g/mole;
(ii) adding an aqueous, possibly buffered, solution of oligonucleotides or polynucleotides to the aqueous solution from step (i);
(iii) freezing the aqueous solution from step (ii) and subsequently freeze-drying the frozen solution to obtain a freeze-dried substrate;
(iv) providing a solution of the hydrophobic biodegradable polymer in an organic solvent;
(v) adding the substrate obtained according to step (iii) to the solution of the polymer; and
(vi) removing the organic solvent to obtain the composite.

2. The method of claim 1, wherein the aqueous solution from step (i) contains polyethylene glycol in 1.2 to 100 fold gravimetric excess to the oligonucleotide or polynucleotide.

3. The method of claim 1, wherein the oligonucleotides or polynucleotides comprise DNA fragments.

4. The method of claim 1, wherein the hydrophobic biodegradable polymer is a polylactide, in particular, poly-L-lactide.

5. The method of claim 1, wherein the organic solvent from step (iv) is chloroform.

6. A composite obtained or obtainable according to a method comprising the steps of:
(i) producing an aqueous solution of a polyethylene glycol having a mean molecular weight in the range from 600 to 8,000 g/mole;
(ii) adding an aqueous, possibly buffered, solution of oligonucleotides or polynucleotides to the aqueous solution from step (i);
(iii) freezing the aqueous solution from step (ii) and subsequently freeze-drying the frozen solution to obtain a freeze-dried substrate;
(iv) providing a solution of the hydrophobic biodegradable polymer in an organic solvent;
(v) adding the substrate obtained according to step (iii) to the solution of the polymer; and
(vi) removing the organic solvent to obtain the composite.

7. A composite useful as a coating material for a medical implant or as a filler of a cavity of a medical implant, the composite produced according to a method comprising the steps of:
(i) producing an aqueous solution of a polyethylene glycol having a mean molecular weight in the range from 600 to 8,000 g/mole;
(ii) adding an aqueous, possibly buffered, solution of oligonucleotides or polynucleotides to the aqueous solution from step (i);
(iii) freezing the aqueous solution from step (ii) and subsequently freeze-drying the frozen solution to obtain a freeze-dried substrate;
(iv) providing a solution of the hydrophobic biodegradable polymer in an organic
(v) adding the substrate obtained according to step (iii) to the solution of the polymer; and
removing the organic solvent to obtain the composite.

8. The method of claim 3, wherein the DNA fragments are dODN.

9. The method of claim 4, wherein the polylactide is poly-L-lactide.
